(19) United States
(12) Patent Application Publication
(10) Pub. No.: US 2009/0157190 A1
(43) Pub. Date: Jun. 18, 2009

(54) Osteotomy spacer

(75) Inventors: Carlos E. Collazo, Old Greenwich, CT (US); Michael D. Lynch, Skaneateles, NY (US); Alexander Curry, Attleboro, MA (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Inc., Allendale, NJ (US)

(21) Appl. No.: 12/002,002

(22) Filed: Dec. 13, 2007

(51) Int. Cl. A61F 2/38

(52) U.S. Cl. 623/20.32; 623/20.14

(57) ABSTRACT

A device for use in connection with a bone, comprising a first surface and a second surface spaced apart from each other at a predetermined distance and angled relative to each other along a portion thereof. The device having a first ridge projecting from the first surface in a direction away from the second surface, the first ridge extending substantially along a length of the first surface, and a second ridge projecting from the second surface in a direction away from the first surface, the second ridge extending substantially along a length of the second surface in a direction substantially parallel to the first ridge. The device further includes a remote surface positioned more distant from the center of a bone, a proximate surface positioned more near the center of a bone, a fixed end surface, and a removable end surface extending in a first direction. The body of the device extends between the remote surface and the proximate surface in a medial-lateral direction and between the fixed end surface and the removable end surface in an anterior-posterior direction. A first score mark extends between the medial surface and the lateral surface in a direction substantially parallel to the direction of the removable end surface.

### BACKGROUND OF THE INVENTION

High tibial osteotomy (“HTO”) procedures have become a well-established means of treating unicompartmental degenerative arthritis of the knee. This condition occurs due to uneven weight bearing of the femoral condyles on either the medial or lateral joint compartments of the tibia. Such uneven weight bearing results from either a varus or valgus defect in the tibia. A varus or valgus defect occurs when the knee joint shifts either medially (valgus) or laterally (varus) with respect to the mechanical axis. It is generally accepted that the preferred location for the mechanical axis of the knee is at about 62% of the tibial plateau from medial to lateral. The process for determining the location of the mechanical axis is known in the art. A varus deformity generally results in increased loading on the medial joint compartment, while a valgus defect results in increased loading on the lateral joint compartment. A high-tibial osteotomy procedure uses one of various techniques to bring the knee into proper mechanical alignment by correcting a deformity therein, whether varus or valgus.

One existing high-tibial osteotomy procedure is the opening wedge HTO. In this procedure, a single cut is made from, for example, the medial cortex of the tibia across to near the lateral cortex in order to correct a varus defect. The cut in an opening wedge HTO procedure extends through almost the entire tibia, leaving only enough bone on the lateral tibia to form a hinge section which serves to keep the tibial plateau connected to the remainder of the bone. The cut is then forced open to form a wedge having an angle corresponding to the required amount of angular correction. This procedure can also be used to correct a valgus defect, with the cut originating on the lateral tibia, extending through the tibia to near the medial tibia. The necessary cut is typically made using a cutting guide, of which various forms are known, affixed to the tibia.

Upon completion of the cut, the cutting guide, should one be used in the procedure, is removed and the bone is typically displaced by inserting two plates into the cut and turning a jackscrew. A metal wedge may also be used to expand the wedge cut by impacting the wedge into the cut and advancing it until the desired amount of correction is achieved. Once the cut is opened, an appropriately shaped spacer can be inserted into the cut to support the tibial plateau at the desired angle. The spacer can be made of a known bone-substitute material, an autograft taken from the patient's iliac crest or an allograft taken from a donor. The wedge is then secured in place using hardware typically in the form of bone plates and screws.

An alternative procedure is what is known as a closing-wedge osteotomy. In such a procedure, a wedge of bone is removed from the tibia, closing the opening left by the removal of the wedge, and securing the bone in its new configuration. The wedge is shaped to correspond to the appropriate amount of angular correction necessary to bring the knee joint into proper alignment. Generally the wedge is shaped so as to span almost the entire medial-lateral width of the tibia, leaving only a narrow “hinge” section of bone on the closed end of the wedge. Once the bone wedge is resected, the opening is forced closed and is typically held in such a position using a staple or other similar device, including bone screws and/or plates. Such procedures are shown in U.S. Pat. No. 5,980,526 to Johnson, et al.; U.S. Pat. No. 6,796,986 to Duffner; U.S. Pat. No. 5,911,724 to Wehrli; U.S. Pat. No. 5,053,039 to Hoffman, et al.; U.S. Pat. No. 5,540,695 to Levy, and; U.S. Pat. No. 5,601,565 to Huebner.

The length of the cut formed in the proximal tibia during both the opening and closing wedge procedures can be problematic due to the large amount of torsional loading that is applied to the tibia during routine movement. Both procedures leave only a narrow section of bone at an outer edge thereof to bear such loads. The narrow section of bone, however, is unlikely to withstand such loads, making fracture of the remaining bone a primary concern. To reduce the likelihood of fracture, fixation hardware is often applied to the opposite side of the tibial plateau, in the area of the bone cut. Such hardware is most often bulky, causing pain and additional trauma to the knee joint during surgery and discomfort during recovery and beyond. The hardware is also often problematic should a subsequent total knee arthroplasty (“TKA”) procedure be performed, and must often be removed, further complicating this procedure and reintroducing an area of weakness to the location of the osteotomy procedure.

Therefore, it is desirable to provide a device to provide stability to the tibial plateau after an osteotomy procedure while maintaining a reduced amount of hardware.

### SUMMARY OF THE INVENTION

This invention relates to an implant to be used in an open wedge tibial osteotomy to sufficiently stabilize the correction while natural healing of the bone takes place.

One aspect of this invention is the inherent stability that it provides to the reconstruction. By partially filling the gap created by the correction, it allows compressive loads to be transmitted from the tibial plateau, through the implant, and onto the underlying distal bone. Its two ribs, one proximal and one distal, allow torsional loads to be shared by the implant. Providing compressive and torsional stability lessens the loading demand on a bone plate if one were to be used. This allows the use of a much smaller, less invasive plate to supplement the implant.

A second aspect of this invention is the ability to intraoperatively cut its length to the appropriate size to match the bone. This is made possible by providing score marks at predetermined lengths and employing a cutter to “shear” the implant along those marks. This would significantly reduce the amount of inventory necessary to accommodate the size variation that exists from patient to patient.

A third aspect of the invention is providing for long term biologic fixation. The implant can be made either wholly or partially out of materials with surfaces known in the art to enable bony ingrowth. These materials may include Cobalt Chrome porous coating or Titanium foam, either uncoated or coated with osteoconductive materials such as hydroxy apatite (“HA”) or tricalcium phosphate (“TCP”). Other osteoconductive materials may include resorbable nanoceramics.

A fourth aspect of this invention is providing a pathway through the implant for materials to be injected through the implant and into the adjacent cancellous bone. This material, such as polymethyl methacrylate (“PMMA”) bone cement or ultrasonically melted polylactic acid (“PLA”) can be used for immediate fixation obtained intraoperatively. Osteogenic materials, such as bone marrow aspirate may also be used to promote healing.

This invention provides added stability to the correction and reduces incidence of plate failure with a much smaller, less invasive plate. Alternate additional cement fixation may provide sufficient stability to eliminate the use of a metal plate altogether. A resorbable implant reduces the amount of “metal” hardware needed and, in addition, biologic fixation provides additional stability to the reconstruction. One long implant length, which can be reduced as desired, reduces the amount of costly inventory. The above and various other aspects of this invention are exemplified by a series of preferred embodiments.

One embodiment of the present invention relates to a device for use in connection with a bone. The device may include a first surface and a second surface spaced apart from each other at a predetermined distance. There may be a first ridge projecting from the first surface in a direction away from the second surface, the first ridge extending substantially along a length of the first surface. There may also be a second ridge projecting from the second surface in a direction away from the first surface, the second ridge extending substantially along a length of the second surface in a direction substantially parallel to the first ridge.

The device may further include an anterior face and a posterior face, wherein the first ridge and the second ridge each extend from near the anterior face to near the posterior face. Preferably, the device may include a remote surface, positioned more distant from the center of a bone, a proximate surface positioned more near the center of a bone, a fixed end surface and a removable end surface. The body of the device may extend between the remote surface and the proximate surface in a medial-lateral direction and the fixed end surface and the removable end surface in an anterior-posterior direction. The first score mark may extend between the remote surface and the proximate surface in a direction substantially parallel to the removable end surface. The fixed end surface may preferably be angled relative to the removable end surface.

In a preferred embodiment, the first surface and the second surface may be angled relative to each other along portions thereof at an angle that substantially matches an angle to be formed in a bone during a bone osteotomy procedure. The first ridge and/or the second ridge may be substantially semi-circular in shape. Furthermore, in one preferred embodiment, the first and second ridges may extend beyond the plane of the proximate surface in a medial-lateral direction.

The device of the present invention may also include a first ridge with an outside surface made of a porous material. Alternatively, the outside surface of the first ridge may also be coated with an osteoconductive material.

In an alternative embodiment, the device for use in connection with a bone may include a body defined by first and second surfaces and extending therebetween in a proximal-distal direction. The body may have a predetermined thickness, wherein the first surface has a first score mark formed therein.

In a further embodiment of the device, the first score mark may define a first removable portion of the device. A second score mark may also be present, which second score mark may define a second removable portion of the device. Preferably, the first score mark may be one of a plurality of score marks, defining a plurality of removable portions of the device, the plurality including the first removable portion.

In an alternative embodiment, the device may include an anterior surface and a posterior surface, the anterior surface and the posterior surface including a bore formed therebetween. The bore may include a first channel open to the first surface and/or a second channel open to the second surface. The first channel may further open to the first surface in the area of the first ridge.

A further embodiment of the present invention relates to a method of performing a bone osteotomy procedure. This method may include the steps of forming a hole at a predetermined location in a bone and forming a cut along a predetermined path in the bone with the cut intersecting the hole. The method may further include forcing the cut open to form an opening in the bone and inserting a spacer into the opening. The spacer may include a first rib and a second rib, and the opening may include a first groove formed by a first portion of the hole and a second groove formed by a second portion of the hole. The spacer may be inserted into the opening such that the first rib extends into the first groove and the second rib extends into the second groove.

In a further embodiment of the method, the spacer may include a channel open to an end surface and an upper surface thereof, and the step of inserting the spacer into the opening may include positioning the upper surface of the spacer so as to contact a first portion of the bone. The method may further include the step of applying a bone cement into the channel. This method may further include a channel open to a lower surface of the spacer, wherein the step of inserting the spacer into the opening includes positioning the upper surface of the spacer so as to contact a second portion of the bone.

In an alternative embodiment, the spacer may include a first score mark formed on an outside surface thereof, the first score mark defining a first removable portion of the spacer. The method may further include determining an appropriate length for the spacer and optionally removing the first removable portion of the spacer based on the appropriate length for the spacer. Preferably, the spacer may include a plurality of score marks formed on an outside surface thereof, the plurality of score marks defining a plurality of removable portions of the spacer. Further preferably, the method may include the step of determining an appropriate length for the spacer and selectively removing at least one of the removable portions based on the determination of the appropriate length for the spacer.

A further embodiment of the present invention relates to a kit for use in connection with a bone osteotomy procedure. The kit may include a spacer having a plurality of outside surfaces. The plurality of outside surfaces may include an upper surface and a lower surface spaced apart from each other at a predetermined distance. The spacer may include a first score mark formed on one of the outside surfaces, the first score mark defining a first removable portion of the spacer. The kit may further include a cutting device adapted to remove the first removable portion from the spacer.

In an alternative embodiment of the kit, the cutting device may include a body having a channel, the channel having a width suitable for securing the spacer therein. The cutting instrument may further include a blade affixed to the body so as to be moveable along a predetermined path through the score mark of the spacer. Preferably, the first score mark of the spacer may be one of a plurality of score marks and the first removable section of the spacer may be one of a plurality of removable sections defined by the score marks. Further preferably, the channel may be shaped so as to slideably receive the spacer therein such that one of the score marks can be positioned substantially within the predetermined path of the blade.

As used herein when referring to bones or other parts of the body, the term “proximal” means close to the heart and the term “distal” means more distant from the heart. The term “anterior” means toward the front part or the face and the term “posterior” means toward the back of the body. The term “medial” means toward the midline of the body and the term “lateral” means away from the midline of the body.

### BRIEF DESCRIPTION OF THE DRAWINGS

The present invention will be better understood on reading the following detailed description of nonlimiting embodiments thereof, and on examining the accompanying drawings, in which:

FIG. 1 is an anterior view of the proximal end of a tibia having a spacer according to an embodiment of the present invention installed therein;

FIG. 2 is an isometric view of a spacer according to the embodiment of FIG. 1;

FIG. 3 is an anterior view of a proximal tibia during a step of an osteotomy procedure according to an aspect of the present invention;

FIG. 4 is an isometric view of a spacer according to a further embodiment of the present invention;

FIG. 5 is a top view of the spacer shown in FIG. 4;

FIG. 6 is an isometric view of the spacer of FIG. 4 mounted in a cutting instrument to be used with the spacer;

FIG. 7 is an isometric view of a spacer according to a further embodiment of the present invention;

FIG. 8 is a cross-section view of the spacer of FIG. 7;

FIG. 9 is an elevational side view of a spacer according to a further embodiment of the present invention; and

FIG. 10 is an isometric view of the spacer of FIG. 9.

### DETAILED DESCRIPTION

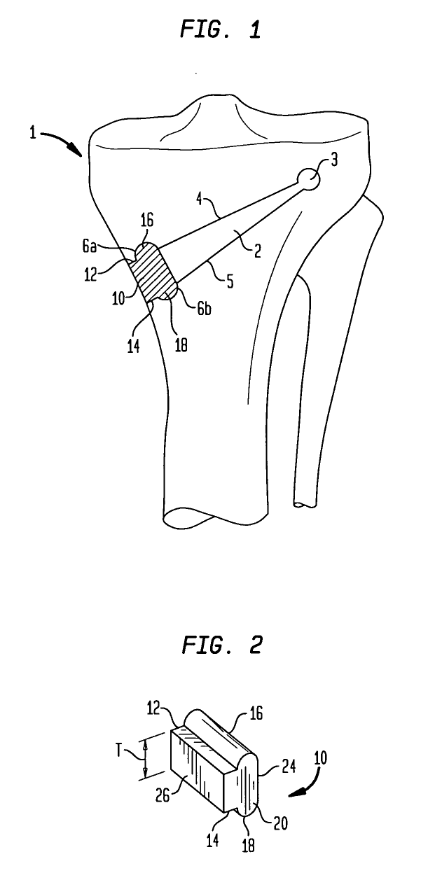

Referring now to the drawings wherein like reference numerals indicate similar features, there is shown in FIG. 1 an anterior view of a tibia 1 with an osteotomy spacer 10 according to an exemplary embodiment of the present invention implanted therein. Spacer 10 is shown implanted in the proximal portion of tibia 1 to secure a wedge formed during an opening-wedge high tibial osteotomy (“HTO”) procedure. The tibia 1 shown in FIG. 1 has been subjected to an opening-wedge HTO procedure on the medial side thereof, but it is contemplated that a spacer according to an embodiment of the present invention can be used in connection with an opening-wedge HTO procedure carried out on the lateral side of the bone or in connection with a closing-wedge HTO procedure performed on either the medial or lateral side of the bone. Additionally, spacer 10 of the present invention can be used in connection with other osteotomy procedures carried out on various bones or in other orthopedic procedures. Spacer 10 may be made either wholly or partially out of materials with surfaces known in the art to enable bony ingrowth, including cobalt chrome porous coating or titanium foam. These materials may further be coated with osteoconductive materials such as HA or TCP. Other osteoconductive materials may include resorbable nanoceramics.

In FIGS. 1 and 2, spacer 10 is shown as having an upper surface 12 and a lower surface 14 spaced apart from each other. Upper surface 12 has a portion thereof that extends upwardly, preferably in a semi-circular shape, to form an upper rib 16. Similarly, lower surface 14 has a portion thereof that extends downwardly to form a lower rib 18. Preferably, both upper rib 16 and lower rib 18 extend along the entire longitudinal length of the spacer, as shown in FIG. 2. As further shown in FIGS. 1 and 2, a portion of upper surface 12 and a portion of lower surface 14 are substantially planar and extend substantially parallel to each other in an anterior-posterior direction, and may be angled relative to each other in a medial-lateral direction to match the angle of opening 2. Although upper surface 12 and lower surface 14 are shown in FIG. 1 to be positioned remotely from the center of the bone with respect to upper and lower ribs 16 and 18, surfaces 12 and 14 may alternatively be positioned proximate the center of the bone.

As shown in FIG. 1, spacer 10 may be used in securing an opening 2 formed during an opening-wedge osteotomy procedure. Preferably, specific spacer 10 is selected to correspond to the appropriate size for opening 2, which can be done prior to surgery or intraoperatively. Further preferably, the relative angle at which the planar portions of upper surface 12 and lower surface 14 are positioned, with respect to the proximal surface of the tibia 1, substantially corresponds to the angle of opening 2. By doing so, the substantially planar portions of upper surface 12 and lower surface 14 help to prevent rotation of spacer 10 about its long axis and maintain a portion of the loading to which the tibia is subjected, thereby helping to maintain the angle of the opening.

When spacer 10 is in place in proximal tibia 1, upper rib 16 fits into an appropriately sized upper groove 6a that is formed in the bone defining upper surface 4 of the opening 2. Similarly, lower rib 18 fits into an appropriately sized lower groove 6b that is formed in the bone defining lower surface 5 of opening 2. The extension of ribs 16, 18 into grooves 6a, 6b aides in both securing spacer 10 within opening 2 and handling torsional loading of the tibia.

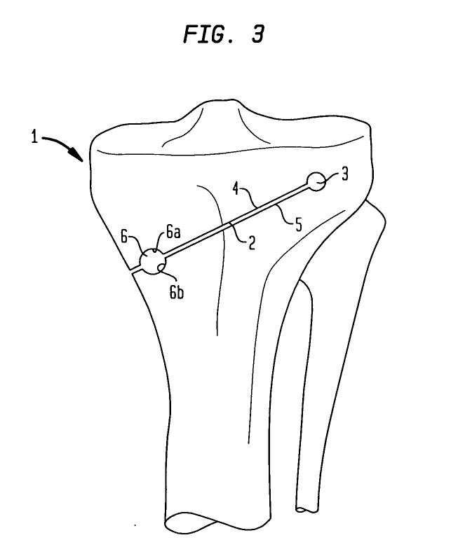

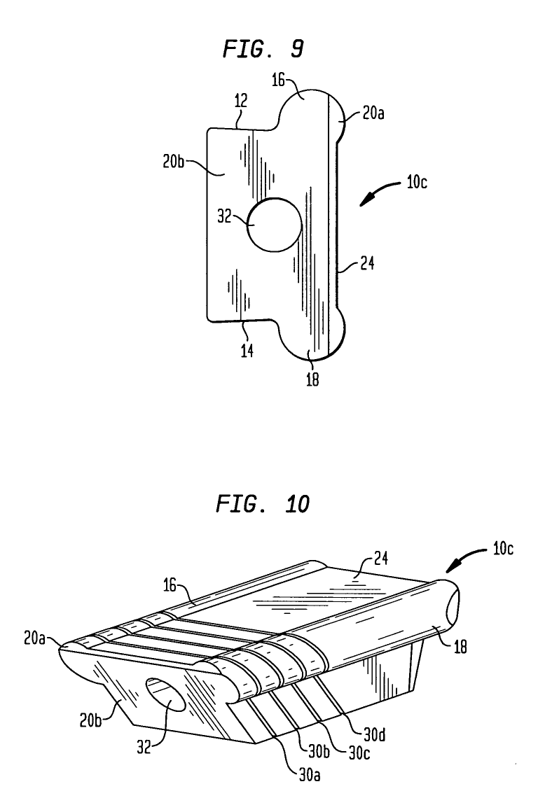

Referring to FIG. 3, there is shown tibia 1 during a step of an osteotomy procedure. A medial hole 6 is bored in the bone at a predetermined medial location and a lateral hole 3 is bored in the bone at a predetermined lateral location. A cut is thereafter made, intersecting medial hole 6 and lateral hole 3, and extending from the medial cortex across to the lateral cortex. Preferably, the cut extends through almost the entire tibia, leaving only enough bone to form a hinge section which serves to keep the tibial plateau connected to the remainder of the bone. The cut may be forced open during an open-wedge osteotomy procedure thereby forming opening 2 and positioning upper groove 6a to receive upper rib 16 of spacer 10 and lower groove 6b to receive lower rib 18 of spacer 10. The spacer may thereafter be positioned within the cut as shown in FIG. 1 and the cut may be closed around the spacer. Alternatively, as where ribs 16 and 18 extend more than 180° (shown in FIGS. 9-10), the spacer may be introduced into the cut by sliding the spacer into place in an anterior-posterior direction.

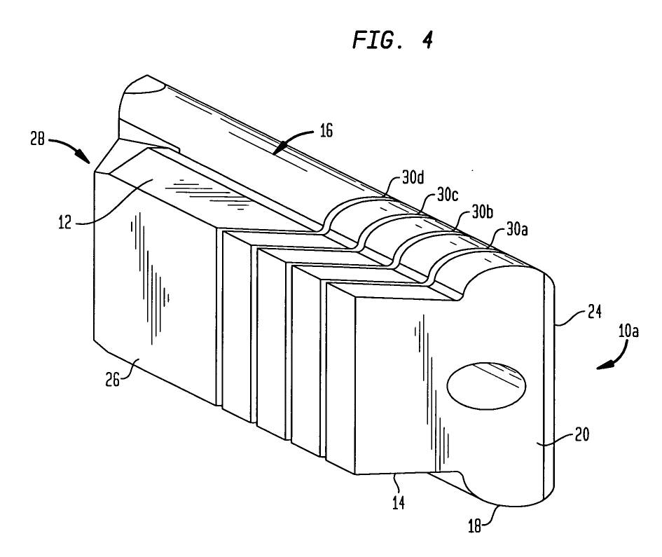

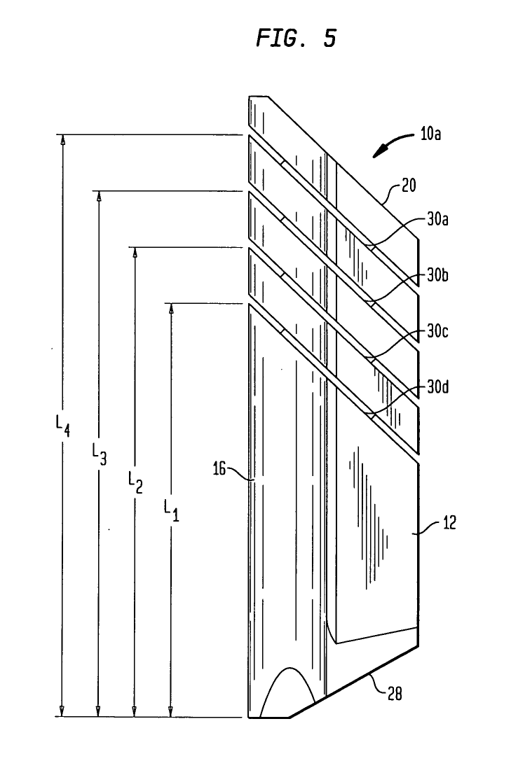

FIGS. 4-5 show an alternate spacer embodiment 10a. Upper surface 12 and lower surface 14 of spacer 10a are shown to extend substantially parallel to each other in an anterior/posterior direction. Remote surface 26 and proximate surface 24 connect upper surface 12 and lower surface 14 and extend longitudinally therebetween. Preferably, spacer 10a also has a fixed end surface 28 and a removable end surface 20. In one embodiment, the fixed end surface 28 may be angled relative to the removable end surface 20 to match the anatomy of a tibia.

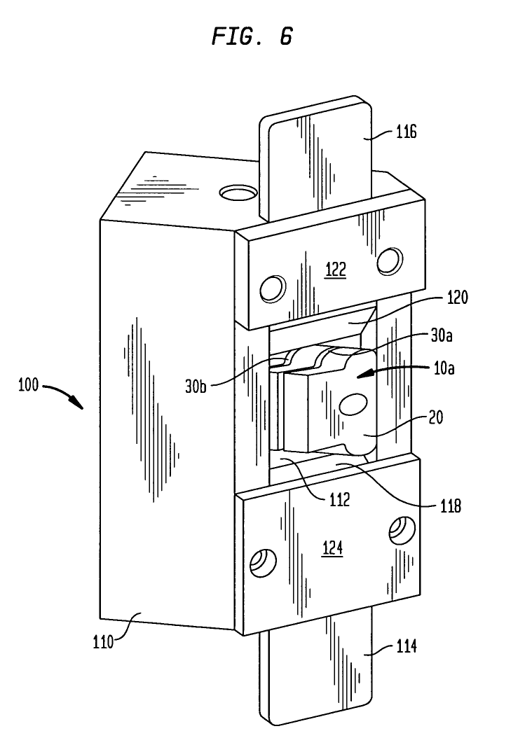

In a preferred embodiment, score marks 30a-30d are provided at predetermined locations along the longitudinal length of spacer 10a. Each score mark 30a-30d extends between remote surface 26 and proximate surface 24 in a direction substantially parallel to removable end surface 20. Each score mark further defines a removable portion of spacer 10, which removable portion may be sheared with a cutting tool 100, shown in FIG. 6. The removal of portions of spacer 10 provides the ability to intraoperatively cut the length L1-L4 of spacer 10 to the appropriate size to match the anterior to posterior dimension of opening 2 into which spacer 10 may be introduced.

Referring to FIG. 6, there is shown a cutting tool 100 for cutting a spacer 10a along score marks 30a-30d. The cutting tool has a body 110 with a channel 112 through which spacer 10a can pass. There is attached to a face of body 110 blade guides 122, 124 positioned on either side of channel 112. Adjacent to blade guides 122, 124 are blades 118, 120 connected to blade handles 114, 116. Blades 118, 120 are each positioned on opposite sides of channel 112 through which spacer 10a passes. It is thereby possible for a user of cutting tool 100, by pressing blade handles 114, 116 inwardly toward channel 112, to move blades 118, 120 along a predetermined path through a score mark 30a-30d of spacer 10a. The ability to intraoperatively cut spacer 10a to a desired length allows storage of one implant length at the facility where surgery is performed and thereby reduces the amount of costly inventory.

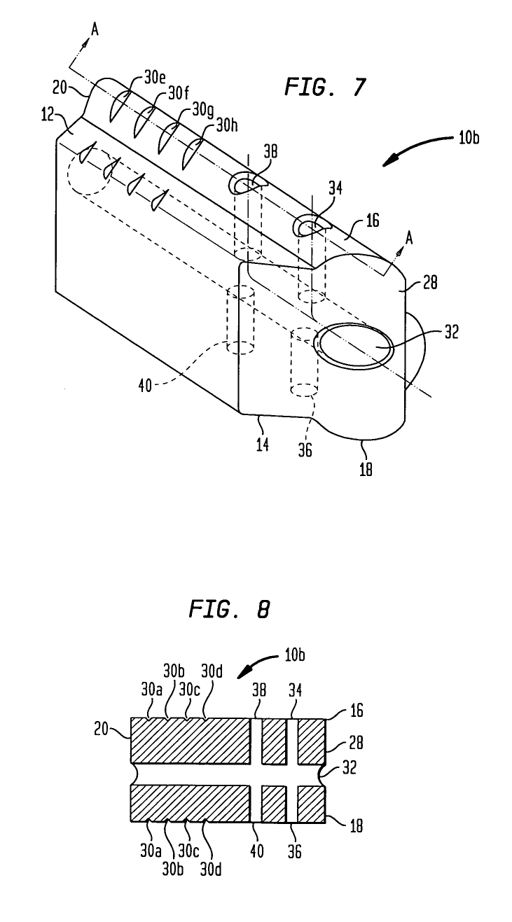

Referring to FIGS. 7-8 there is shown an alternate spacer embodiment 10b wherein a hole 32 is formed between anterior face 20 and posterior face 28 of spacer 10b. Preferably, hole 32 forms channels 34, 38 opening to upper surface 12 and channels 36, 40 opening to lower surface 14. The channels form pathways through spacer 10b for injection of materials into the adjacent cancellous bone while spacer 10b is seated in opening 2. Materials that may be injected include PMMA bone cement or ultrasonically melted PLA. These materials may provide immediate intraoperative fixation.

Referring to FIGS. 9-10, there is shown a further spacer embodiment 10c wherein upper surface 12 has a portion thereof that extends upwardly, preferably in a semi-circular shape, to form an upper rib 16. Upper rib 16 preferably extends beyond the plane of proximate surface 24, thereby forming a protrusion from proximate surface 24 adjacent surface 12. Similarly, lower surface 14 has a portion thereof that extends downwardly to form a lower rib 18. Lower rib 18 preferably extends beyond the plane of proximate surface 24, thereby forming a protrusion from proximate surface 24 adjacent surface 14. In one embodiment upper rib 16 and/or lower rib 18 may have a circumference of 225°. One purpose of extended ribs 16 and 18 is to increase rotational stability of the implant while the implant is seated in the bone. Preferably, both upper rib 16 and lower rib 18 extend along the entire longitudinal length of the spacer, as shown in FIG. 10.

FIG. 10 further shows the configuration of surface 20a and surface 20b of alternate spacer embodiment 10c. Surface 20a is preferably positioned perpendicular to proximate surface 24. Surface 20a also preferably includes a lateral portion of upper rib 16 and lower rib 18. Surface 20b is angled relative to surface 20a to conform to the anatomy of a tibia.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A device for use in connection with a bone, comprising:
a first surface and a second surface spaced apart from each other at a predetermined distance;
a first ridge projecting from the first surface in a direction away from the second surface, the first ridge extending substantially along a length of the first surface;
a second ridge projecting from the second surface in a direction away from the first surface, the second ridge extending substantially along a length of the second surface in a direction substantially parallel to the first ridge;
a remote surface positioned more distant from the center of a bone;
a proximate surface positioned more near the center of a bone;
a fixed end surface;
and a removable end surface extending in a first direction;
wherein the body of the device extends between the remote surface and the proximate surface in a medial-lateral direction and the body of the device extends between the fixed end surface and the removable end surface in an anterior-posterior direction, and wherein a first score mark extends between the remote surface and the proximate surface in a direction substantially parallel to the direction of the removable end surface.

2. The device of claim 1, wherein the first surface and the second surface are angled relative to each other along portions thereof at an angle that substantially matches an angle to be formed in a bone during a bone osteotomy procedure.

3. The device of claim 1, wherein the first ridge is substantially semi-circular in shape.

4. The device of claim 1, wherein the second ridge is substantially semi-circular in shape.

5. The device of claim 1 wherein the first and second ridges extend beyond the plane of said proximate surface in a medial-lateral direction.

6. The device of claim 1 further including an anterior face and a posterior face, wherein the first ridge and the second ridge each extend from near the anterior face to near the posterior face.

7. The device of claim 1, wherein the first ridge includes an outside surface made from a porous material.

8. The device of claim 1, wherein the first ridge includes an outside surface coated with an osteoconductive material.

9. The device of claim 1 further including a body defined by the first and second surfaces and extending therebetween in a proximal-distal direction, the body having a predetermined thickness, wherein the first surface has a first score mark formed therein.

10. The device of claim 1, wherein the fixed end surface is angled relative to the removable end surface.

11. The device of claim 1, wherein the first score mark defines a first removable portion of the device.

12. The device of claim 11, further including a second score mark, wherein the second score mark defines a second removable portion of the device.

13. The device of claim 11, wherein the first score mark is one of a plurality of score marks, defining a plurality of removable portions of the device, the plurality including the first removable portion.

14. The device of claim 1 further including an anterior surface and a posterior surface, wherein said anterior surface and said posterior surface include a bore formed therebetween, the bore including a first channel open to the first surface.

15. The device of claim 14, wherein the bore includes a second channel open to the second surface.

16. The device of claim 14, wherein the first channel is open to the first surface in the area of the first ridge.

17. A method of performing a bone osteotomy procedure comprising the steps of:
forming a hole at a predetermined location in a bone;
forming a cut along a predetermined path in said bone, said cut intersecting said hole;
forcing said cut open to form an opening in said bone including said hole; and
inserting a spacer into said opening, said spacer including a first rib and a second rib;
wherein the opening includes a first groove formed by a first portion of said hole and a second groove formed by a second portion of said hole, and wherein said spacer is inserted into said opening such that the first rib extends into the first groove and the second rib extends into the second groove.

18. The method of claim 17, wherein the spacer further includes a channel open to an end surface thereof and open to an upper surface thereof, and wherein the step of inserting the spacer into the opening includes positioning the upper surface of the spacer so as to contact a first portion of the bone, the method further including the step of applying a bone cement into the channel.

19. The method of claim 18, wherein the channel is further open to a lower surface of the spacer, wherein the step of inserting the spacer into the opening includes positioning the upper surface of the spacer so as to contact a second portion of the bone.

20. The method of claim 17, wherein the spacer includes a first score mark formed on an outside surface thereof, the first score mark defining a first removable portion of the spacer, the method further including determining an appropriate length for the spacer and optionally removing the first removable portion of the spacer based on the appropriate length for the spacer.

21. The method of claim 17, wherein the spacer includes a plurality of score marks formed on an outside surface thereof, the plurality of score marks defining a plurality of removable portions of the spacer, the method further including the step of determining an appropriate length for the spacer and selectively removing at least one of the removable portions based on the determination of the appropriate length for the spacer.

22. A kit for use in connection with a bone osteotomy procedure, comprising:
a spacer including a plurality of outside surfaces, said plurality of outside surfaces including an upper surface and a lower surface, said upper and lower surfaces being spaced apart from each other at a predetermined distance, the spacer including a first score mark formed on one of the plurality of outside surfaces, the first score mark defining a first removable portion of the spacer; and
a cutting device adapted to remove the first removable portion from the spacer.

23. The kit of claim 22, wherein the cutting device includes a body having a channel therein, the channel having a width suitable for securing the spacer therein, the cutting instrument further including a blade affixed to the body so as to be moveable along a predetermined path through the score mark of the spacer.

24. The kit of claim 23, wherein the first score mark of the spacer is one of a plurality of score marks and wherein the first removable section of the spacer is one of a plurality of removable sections defined by the plurality of score marks.

25. The kit of claim 24, wherein the channel is shaped so as to slideably receive the spacer therein such that one of the plurality of score marks can be positioned substantially within the predetermined path of the blade.
